(19) United States
(12) Patent Application Publication
(10) Pub. No.: US 2025/0110195 A1
(43) Pub. Date: Apr. 3, 2025

(54) Recording Diffusion-Weighted Measurement Data of an Examination Object Using a Magnetic Resonance System

(71) Applicant: Siemens Healthineers AG, Forchheim (DE)

(72) Inventor: Mario Zeller, Erlangen (DE)

(73) Assignee: Siemens Healthineers AG, Forchheim (DE)

(21) Appl. No.: 18/895,753

(22) Filed: Sep. 25, 2024

(30) Foreign Application Priority Data
Sep. 29, 2023 (DE) 10 2023 209 641.2

(51) Int. Cl. G01R 33/563; A61B 5/055

(52) U.S. Cl. CPC G01R 33/56341; A61B 5/055

(57) ABSTRACT

A plurality of diffusion-weighted measurement datasets (including at least one initial and one further measurement dataset) are recorded following a common excitation and thus within a repetition time (TR) using segmented recording in the readout direction.

### TECHNICAL FIELD

The disclosure relates to an optimized recording of diffusion-weighted measurement data of an examination object by means of a magnetic resonance system.

### BACKGROUND

Magnetic resonance technology (hereinafter, the abbreviation MR stands for magnetic resonance) is a known technology with which images of the interior of an examination object can be generated. Expressed simply, for this purpose, the examination object is positioned in a magnetic resonance device in a relatively strong, static, homogeneous main magnetic field, also known as the B₀ field, with field strengths of 0.2 tesla to 7 tesla or more, so that its nuclear spins become oriented along the main magnetic field. In order to trigger nuclear spin resonances that are measurable as signals, high-frequency excitation pulses (RF pulses) are radiated into the examination object, and the nuclear spin resonances produced are measured as so-called k-space data and; on the basis thereof, MR images are reconstructed, or spectroscopic data is established. For position encoding of the measurement data, rapidly switched magnetic gradient fields, known as gradients for short, are overlaid on the main magnetic field. A scheme that is used that defines a temporal sequence of RF pulses to be radiated in and gradients to be switched is known as a pulse sequence (scheme) or sequence for short. The recorded measurement data is digitized and stored as complex number values in a k-space matrix. From the k-space matrix occupied with values, an associated MR image can be reconstructed, for example, by means of a multi-dimensional Fourier transform.

In a magnetic resonance system, the measurable volume of a magnetic resonance tomography recording is restricted in all three spatial directions due to physical and technical conditions, such as a limited magnetic field homogeneity and a non-linearity of the gradient field. Therefore, a recording volume, a so-called field of view (FoV), is restricted to a volume in which the aforementioned physical features lie within a predetermined tolerance range, and thus, a faithful imaging of the object under examination with the usual scan sequences is possible. The field of view delimited in this way is, however, significantly smaller, in particular in the x and y-directions, i.e., perpendicularly to a longitudinal axis of a tunnel of the magnetic resonance system, than the volume defined by the annular tunnel of the magnetic resonance system. In typical magnetic resonance systems, a diameter of the annular tunnel is, for example, approximately 60 cm, whereas the diameter of the typically used field of view in which the aforementioned physical features are within the tolerance range is approximately 50 cm.

Usually, a magnetic resonance recording is composed of a large number of individual partial measurements in which raw data is acquired from different slices of the examination object in order subsequently to reconstruct volume image data therefrom.

Furthermore, however, in many investigations, it is also necessary to carry out a plurality, i.e., a whole series of magnetic resonance recordings of the examination object, wherein a particular measurement parameter is varied. Using the measurements, the effect of this measurement parameter on the examination object is observed in order later to draw diagnostic conclusions therefrom. A series should be understood as being at least two, but usually more than two, magnetic resonance recordings. Usefully therein, a measurement parameter is varied such that the contrast of a particular material type excited during the measurements, for example, a tissue type of the examination object or a chemical substance which is significant for most, or for particular, tissue types, for example, water, is influenced as strongly as possible by the variation of the measurement parameter. This ensures that the effect of the measurement parameter on the examination object is particularly visible.

A typical example of a series of magnetic resonance recordings under the variation of a measurement parameter strongly influencing the contrast is a so-called diffusion weighting imaging (DWI) method. Diffusion should be understood to be the Brownian motion of molecules in a medium. During diffusion weighting imaging, typically, a plurality of images with different diffusion directions and weightings are acquired and combined with one another. The strength of the diffusion weighting is usually defined by the so-called b-value. The diffusion images with different diffusion directions and weightings and/or the images combined therefrom can then be used for diagnostic purposes. Thus, with suitable combinations of the diffusion-weighted images recorded, parameter maps with particular diagnostic significance can be created, for example, maps that represent the “apparent diffusion coefficient” (ADC) or the “fractional anisotropy” (FA). The diffusion imaging is often based upon echoplanar imaging (EPI) due to the shorter acquisition time of the EPI sequence per image and its robustness in relation to movement.

In diffusion-weighted imaging, additional gradients that reveal the diffusion direction and weighting are inserted into a pulse sequence to make visible or measure the diffusion properties of the tissue. These gradients have the effect that tissue with rapid diffusion (e.g., cerebrospinal fluid (CSF)) is subject to a stronger signal loss than tissue with slow diffusion (e.g., the grey matter tissue of the brain). The resultant diffusion contrast is becoming ever more significant clinically, and nowadays, applications go far beyond the classic early identification of ischemic stroke.

Thus, MR diffusion weighting imaging is an important method both for clinical diagnostics and also in fundamental medical research for making microstructural properties and changes visible in biological tissue. As the complexity of the fundamental modeling methods increases, the need for recorded data grows—for example, recordings with a large number of different diffusion encodings (weightings, directions, variations over time, etc.)—and thus, the need to accelerate the measurements in order to be able to record all the desired data in a timespan that is acceptable, for example, for clinical processes.

Methods for accelerating MR recording techniques such as parallel imaging (PAT), compressed sensing (CS), deep learning (DL), or simultaneous multislice (SMS) imaging are known, which, for example, reduce the number of measurement data items to be recorded and reduce the time required for the recording of the measurement data.

Despite the introduction of such acceleration techniques, the recording times of many DWI measurements are still in the region of several minutes, so every approach to further measurement time reduction—ideally in combination with at least one of the aforementioned acceleration techniques—is significant.

Diffusion imaging is often based on EPI recording techniques described below, in particular upon RESOLVE techniques, which are extended with a diffusion preparation due to the short acquisition time per image and their robustness in relation to movement.

Such EPI (echo-planar imaging) recording techniques are among the fastest-known MR recording techniques. In EPI recording techniques, following an RF excitation pulse, an oscillating, that is bipolar, readout gradient is inserted, which, on each change of the polarization direction of the gradient, refocuses the transverse magnetization as far as the T2* decay allows, and thereby generates a gradient echo. In other words, by way of the switching of the bipolar readout gradient following an RF excitation pulse within the free induction decay (FID) following the excitation, or if additionally, an RF refocusing pulse is radiated in following the RF excitation pulse, within the spin echo generated thereby, an echo sequence of rising and falling gradient echoes with alternating sign is generated. EPI pulse sequences can be utilized as a so-called single-shot method, in which all the measurement data for generating an image of a subvolume, e.g., a slice, of the examination object under investigation is recorded following just one RF excitation pulse.

A spatial resolution that can be produced and can be achieved by means of an EPI technique is typically determined by way of B0 inhomogeneities in the examination object under investigation, e.g., in a patient. They can be caused, for example, by susceptibility jumps at air-tissue or bone-tissue boundary surfaces and lead to local phase accumulations across the echo train that is read out, which is expressed by local geometric distortions in the image. In order to minimize the strength of the geometric distortions, the phase accumulations must therefore be minimized. For this purpose, for example, a temporal length of the read-out echo trains can be kept short, which, however, restricts the spatial resolution or makes the use of parallel imaging techniques necessary.

It is further possible to record the k-space in segments, i.e., to undertake the recording of measurement data in a segmented manner. In principle, the segmentation of the k-space can take place in the phase encoding direction and/or in the readout direction.

The RESOLVE recording technique, first described in the article by Porter and Heidemann entitled “High Resolution Diffusion-Weighted Imaging Using Readout-Segmented Echo-Planar Imaging, Parallel Imaging and a Two-Dimensional Navigator-Based Reacquisition” MRM 62, 2009, pp. 468-475 is a variant of an EPI recording technique in which a segmentation takes place in the readout direction rather than in the phase encoding direction.

Expressed in general terms, in a RESOLVE sequence, for example, following a diffusion preparation, the segment of the k-space that is to be filled with measurement data in the subsequent readout phase is defined by way of a pre-phasing gradient. In the readout phase, by means of a sinusoidal readout gradient, a train of echo signals is recorded as measurement data for the specified segment. By means of a further gradient which has a polarity opposite to that of the pre-phasing gradient and is switched following the readout phase, a return is made in the readout direction to the k-space center again, before a further refocusing pulse is radiated in, which leads to the formation of further echo signals which are recorded, for example, by means of a navigator readout gradient as navigator data of the k-space center. The navigator data thus recorded for each segment can be used to enable a combination of the different segments without artifacts in that a non-linear phase correction of possible phase changes is carried out between the recordings of the measurement data in the individual segments on the basis of the phases of the recorded navigator data, and/or in order to detect segments the measurement data of which is unusable and are to be repeated, as is described in greater detail in the aforementioned article by Porter and Heidemann. Measurement data recorded by means of RESOLVE can also be recorded in the phase encoding direction in an accelerated manner, for example, according to a parallel recording technique, i.e., measurement data is recorded in the phase encoding direction, not along all the k-space lines provided for a complete sampling of the k-space as per Nyquist, but according to the acceleration factor PAT=n, only along every nth k-space line.

FIG. 2 shows an example of a typical sampling scheme in the k-space that is achievable with a RESOLVE pulse sequence scheme. Herein, the axis 51 shows, by way of example, the kx-direction and the axis 53 shows the ky-direction. The kx-direction corresponds here to the readout direction and the ky-direction to the phase encoding direction.

By way of a corresponding segment selection gradient in the readout direction, a first starting point 54 for sampling a segment 63 can be specified. The partial k-space line 55 is acquired while a partial gradient with positive polarity of an EPI-typical readout gradient is switched. The partial k-space line 56 is acquired while a partial gradient with negative polarity of an EPI-typical readout gradient is switched. The displacement lying between the partial k-space lines 55 and 56 in the phase encoding direction is achieved by switching a gradient blip in the phase encoding direction.

The further partial k-space lines 57, 58, 59, 60, 61, and 62 are acquired similarly during the switching of further partial gradients of the EPI-typical readout gradient with alternating polarity.

The partial k-space lines 55 to 62 cover segment 63 of the k-space 52 segmented in the readout direction, for example, into segments 62 to 67.

If another segment selection gradient is used in the readout direction, echo signals of another of the segments 64, 65, 66, or 67 of the k-space 52 can be acquired. In the example shown, segment 65 is the central segment in the k-space since it comprises the k-space center in the readout direction.

In the example shown, the trajectories 68 and 69 (in segments 63 and 64) are shown with a certain spacing in the readout direction. This serves purely for better illustration. Indeed, echo signals can be acquired in such a way that overall, no gaps arise in the acquired k-space in the readout direction, so that segmented but overall complete k-space lines can be sampled in the readout direction. The partial k-space lines contained in the fields 70 and 85, marked by way of example, then complement each other, in each case, to a complete k-space line.

A disadvantage of the RESOLVE sequence consists in the long overall recording time required since for a complete k-space, a plurality of segments must be recorded in each case. The recording of each segment occupies a repetition time TR. In addition, each echo train and, thus, each repetition time TR is prolonged by the recording of the image navigator.

In order to shorten the duration of a recording of a complete measurement dataset, as per Nyquist, under particular conditions, particular measurement data of the complete set is not recorded but is added later. For the recording of an incomplete measurement dataset, less time is needed than for a recording of a complete measurement dataset. A method of this type is, for example, a partial Fourier (PF) technique. In PF techniques, typically not the entire k-space, but only a particular portion of the k-space specified by a PF factor and further determined by way of symmetry observations of the k-space is sampled, that is recorded or measured. The symmetry of the k-space is used in PF techniques to enhance or fill the non-measured portion of the k-space with the aid of different reconstruction methods. In a method known as “zero-filling,” unrecorded regions of the k-space are filled with zeros or null values. This is a very simple method that requires little computation power.

An alternative method for enhancing unrecorded measurement data in PF methods uses a so-called POCS (Projection Onto Convex Sets) algorithm, which estimates missing, that is, unmeasured parts of the k-space of a measurement dataset in an iterative process and thereby ensures data consistency with the actually measured parts of the k-space of the measurement dataset, that is, the actually measured k-space values. In this regard, reference is made, by way of example, to the publication “Implementation and Assessment of Diffusion-Weighted Partial Fourier Readout-Segmented Echo-Planar Imaging” by Robert Frost et al. in Magnetic Resonance in Medicine 68:441-451 (2012). This methodology can lead to improved sharpness or spatial resolution but is not always reliably applicable, for example, dependent upon particular phase variations in the underlying measurement dataset.

Particularly in segmented recording techniques such as, in particular, the aforementioned RESOLVE technique that is susceptible to phase changes, phase inconsistencies contained in the measurement datasets between the individual segments in conjunction with PF techniques can lead to artifacts, in particular so-called ringing artifacts and/or grid-like mesh artifacts.

In the article by Dan et al., “Single-Shot Multi-b-value (SSMb) Diffusion-weighted MRI Using Spin Echo and Stimulated Echoes with Variable Flip Angles,” Proc. Intl. Soc. Mag. Reson. Med. 30; p. 1865, 2022, a conventional echo-planar imaging method is presented in which it is possible, by way of the displacement of magnetization into the longitudinal axis and renewed excitation of this magnetization, to obtain a plurality of diffusion weightings per slice following an excitation. An achievable resolution is further limited by inhomogeneities in the examination object.

An SMS technique can also be used in order to reduce the repetition time TR by way of the simultaneous recording of a plurality of slices. In order to obtain an effective diffusion contrast, however, the minimal TR is restricted to approximately 3 seconds so that a reduction of the repetition time TR is often possible to only a limited extent, such that an advantage achievable through the use of an SMS technique is limited.

### SUMMARY

It is an object of the disclosure to enable a recording of diffusion-weighted measurement data with good quality and the shortest possible overall necessary scan time.

The object is achieved with a method for the optimized recording of diffusion-weighted measurement data of an examination object by means of a magnetic resonance system as claimed in claim 1, a magnetic resonance system as claimed in claim 12, a computer program as claimed in claim 13 and an electronically readable data carrier as claimed in claim 14.

A method according to the disclosure for the optimized recording of diffusion-weighted measurement data of an examination object by means of a magnetic resonance system comprises the steps:
a) radiating in a first RF pulse which tilts a longitudinal magnetization into the transverse plane,
b) switching a diffusion-dephasing gradient of a desired diffusion strength in a desired diffusion direction following the first RF pulse,
c) radiating in a second RF pulse which displaces a portion of the magnetization situated in the transverse plane back again into the longitudinal axis, and leaves another portion of the magnetization situated in the transverse plane therein,
d) switching a first diffusion-rephasing gradient in the desired diffusion direction, which rephases the magnetization remaining in the transverse plane so that after the first diffusion-rephasing gradient, a first echo signal is formed,
e) switching a segment-selection gradient in the readout direction for selection of one of at least three segments,
f) recording the first echo signal in the segment selected by way of the segment selection gradient as measurement data according to an echo-planar recording technique and acquiring it as measurement data of a first measurement dataset,
g) following the recording of the first echo signal, radiating in a third RF pulse which tilts at least a portion of the magnetization displaced back into the longitudinal axis by way of the second RF pulse into the transverse plane,
h) switching a further diffusion-rephasing gradient in the desired diffusion direction, which rephases the magnetization tilted by the third RF pulse into the transverse plane, so that after the further diffusion-rephasing gradient, a further echo signal is formed,
i) switching a further segment-selection gradient in the readout direction for selection of one of at least three segments,
j) recording the further echo signal in the segment selected by way of the further segment selection gradient as measurement data according to an echo-planar recording technique and acquiring it as measurement data of a further measurement dataset,
k) storing and/or further processing the acquired first measurement dataset and acquired further measurement datasets.

By way of the recording segmented in the readout direction according to the disclosure and at least one further echo signal following one common exciting first RF pulse, a plurality of diffusion-weighted measurement datasets (at least one first and one further measurement dataset) are recorded following a common excitation and thus within a repetition time TR. Thereby, a measurement time needed overall for a recording of measurement datasets with all the desired diffusion-weightings can be reduced. Furthermore, an influence of a movement of the examination object under investigation, for example, a breathing of a patient under investigation can be minimized by way of the temporally closely connected recordings according to the disclosure of different measurement datasets with different diffusion weightings.

A magnetic resonance system, according to the disclosure, comprises a magnet unit, a gradient unit, a high frequency unit, and a control apparatus, having a diffusion control unit configured for carrying out a method according to the disclosure.

A computer program, according to the disclosure, implements a method according to the disclosure on a control apparatus when it is executed on the control apparatus. For example, the computer program comprises commands which, when the program is executed by a control apparatus, for example, a control apparatus of a magnetic resonance system, cause this control apparatus to carry out a method according to the disclosure. The control apparatus can be constructed in the form of a computer.

Herein, the computer program can also be available in the form of a computer program product which is directly loadable into a memory store of a control apparatus, having program code means in order to carry out a method according to the disclosure when the computer program product is executed in a computing unit of the computing system.

A computer-readable storage medium, according to the disclosure, comprises commands which, when executed by a control apparatus, for example, a control apparatus of a magnetic resonance system, cause this control apparatus to carry out a method according to the disclosure.

The computer-readable storage medium can be configured as an electronically readable data carrier which comprises electronically readable control information stored thereon, which comprises at least one computer program according to the disclosure and is configured such that, when the data carrier is used in a control apparatus of a magnetic resonance system, it carries out a method according to the disclosure.

The advantages and aspects set out in relation to the method apply accordingly also to the magnetic resonance system, the computer program product and the electronically readable data carrier.

### BRIEF DESCRIPTION OF THE DRAWINGS

Further advantages and details of the present disclosure are disclosed in the exemplary aspects described below and by reference to the drawings. The examples given do not represent any restriction of the disclosure. In the drawings:

FIG. 1 shows a schematic flow diagram of a method according to the disclosure,

FIG. 2 shows an example of a sampling scheme in the k-space that is typical for a RESOLVE pulse sequence scheme.

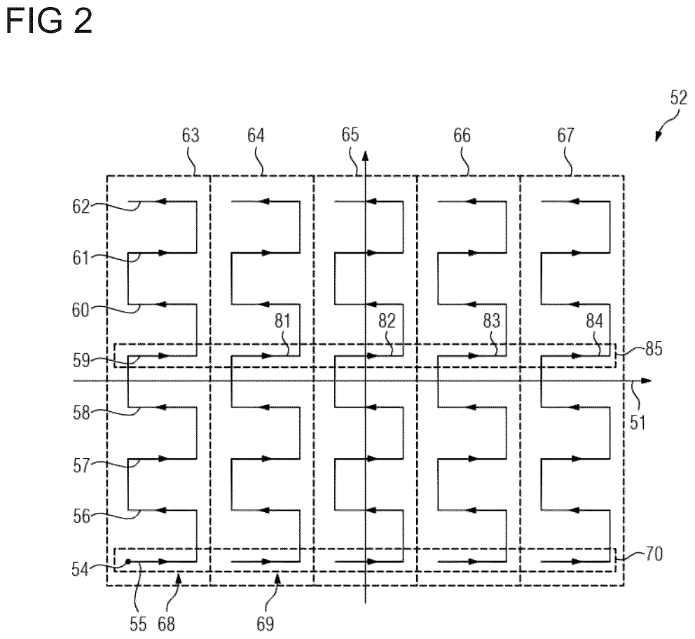

FIG. 3 shows, schematically, a portion of a pulse sequence scheme for the acquisition according to the disclosure of measurement data,

FIG. 4 shows, schematically, a magnetic resonance system according to the disclosure.

### DETAILED DESCRIPTION

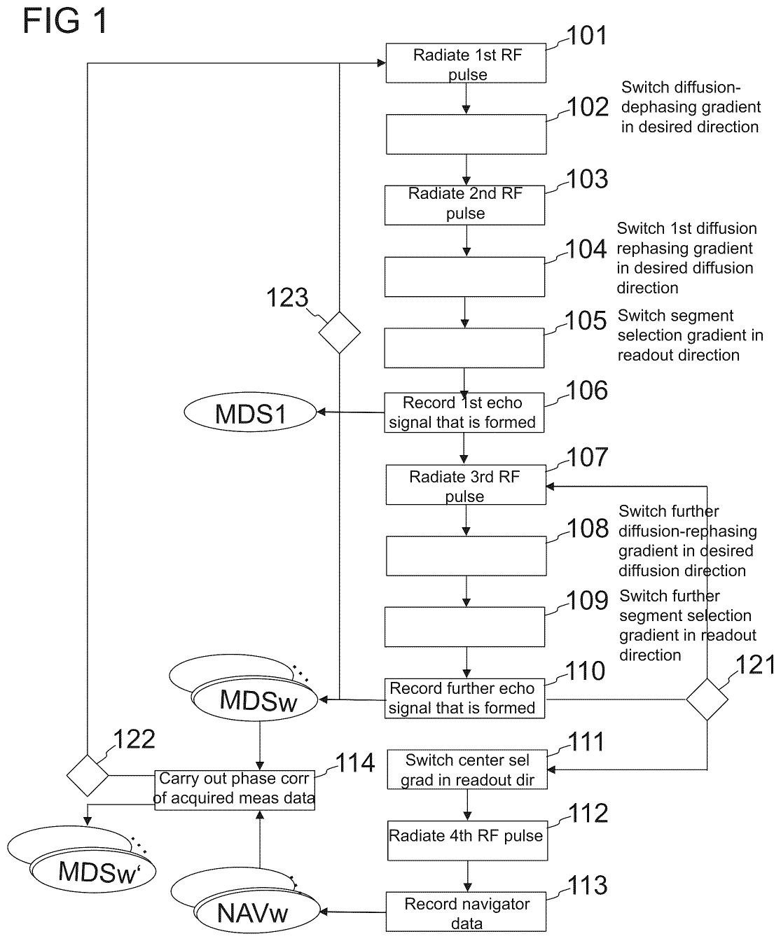

FIG. 1 shows a schematic flow diagram of a method according to the disclosure for the optimized recording of diffusion-weighted measurement data from an examination object by means of a magnetic resonance system.

Therein, a first RF pulse RF1, which tilts a longitudinal magnetization into the transverse plane, is radiated in (block 101).

Following the radiating-in of the RF pulse, a diffusion-dephasing gradient D1 of a desired diffusion strength is switched in a desired diffusion direction (block 102).

A second RF pulse is radiated, which displaces a portion of the magnetization situated in the transverse plane by the first RF pulse back again into the longitudinal axis and leaves another portion of the magnetization situated in the transverse plane therein (block 103). A second RF pulse of this type is known, for example, from the aforementioned article by Dan et al.

A first diffusion-rephasing gradient D2 is switched in the desired diffusion direction, which rephases the magnetization remaining in the transverse plane so that after the first diffusion-rephasing gradient, a first echo signal is formed (block 104).

By way of the switched diffusion-dephasing gradient D1 and the switched first diffusion-rephasing gradient D2, which are both switched before the formation of the first echo signal and had an influence on the magnetization from which the first echo signal is formed, the first echo signal can be diffusion-prepared according to a first diffusion value which is based upon the previously switched diffusion-dephasing gradient D1 and the first diffusion-rephasing gradient D2 and upon the first timespan DT1 elapsed between the switched diffusion-dephasing gradient D1 and the first diffusion-rephasing gradient D2, so that the acquired measurement data is diffusion-weighted according to the first diffusion value.

The first diffusion value (also called the b-value) can herein be determined, for example, according to b=γ² δ² (DT1−δ/3), where γ is the gyromagnetic ratio, G is the gradient amplitude of the diffusion-dephasing gradient D1 and of the first diffusion-rephasing gradient D2, δ is the gradient duration of the diffusion-dephasing gradient D1 and of the first diffusion-rephasing gradient D2 and DT1 is the duration between the pair of diffusion-dephasing gradient D1 and first diffusion-rephasing gradient D2.

A segment selection gradient Se1 is switched in the readout direction for selection of one of at least three segments in which a k-space is to be filled with measurement data (block 105).

The first echo signal that is formed is recorded in the segment selected by way of the segment selection gradient Se1 as measurement data according to an echo-planar recording technique, and the measurement data is acquired as measurement data of a first measurement dataset MDS1 (block 106).

Following the recording of the first echo signal, a third RF pulse RF3 is radiated, which tilts at least a portion of the magnetization displaced by way of the second RF pulse RF2 back into the longitudinal axis into the transverse plane (block 107).

A further diffusion-rephasing gradient D3 is switched in the desired diffusion direction, which rephases the magnetization tilted by the third RF pulse RF3 into the transverse plane so that after the further diffusion-rephasing gradient D3, a further echo signal is formed (block 108).

By way of the switched diffusion-dephasing gradient D1 and the switched further diffusion-rephasing gradient D3, which are both switched before the formation of the further echo signal and influenced the magnetization from which the further echo signal is formed, the further echo signal can be diffusion-prepared according to a further diffusion value which is based upon the previously switched diffusion-dephasing gradient D1 and the further diffusion-rephasing gradient D3 and upon the further timespan DTw elapsed between the switched diffusion-dephasing gradient D1 and the further diffusion-rephasing gradient D3 so that the acquired measurement data is diffusion-weighted according to the further diffusion value.

The further diffusion value (b-value) can herein be determined, for example, according to b=γ² δ² (DTw−δ/3), where γ is the gyromagnetic ratio, G is the gradient amplitude of the diffusion-dephasing gradient D1 and of the further diffusion-rephasing gradient D3, δ is the gradient duration of the diffusion-dephasing gradient D1 and of the further diffusion-rephasing gradient D3 and DTw is the duration between the pair of diffusion-dephasing gradient D1 and further diffusion-rephasing gradient D3.

A further segment-selection gradient Sew is switched in the readout direction for selection of one of the at least three segments (block 109).

The further echo signal that is formed is recorded in the segment selected by way of the further segment selection gradient Sew as measurement data according to an echo-planar recording technique, and the measurement data is acquired as measurement data of a further measurement dataset MDSw (block 110).

Acquired first measurement datasets MDS1 and acquired further measurement datasets MDSw can be stored and/or further processed.

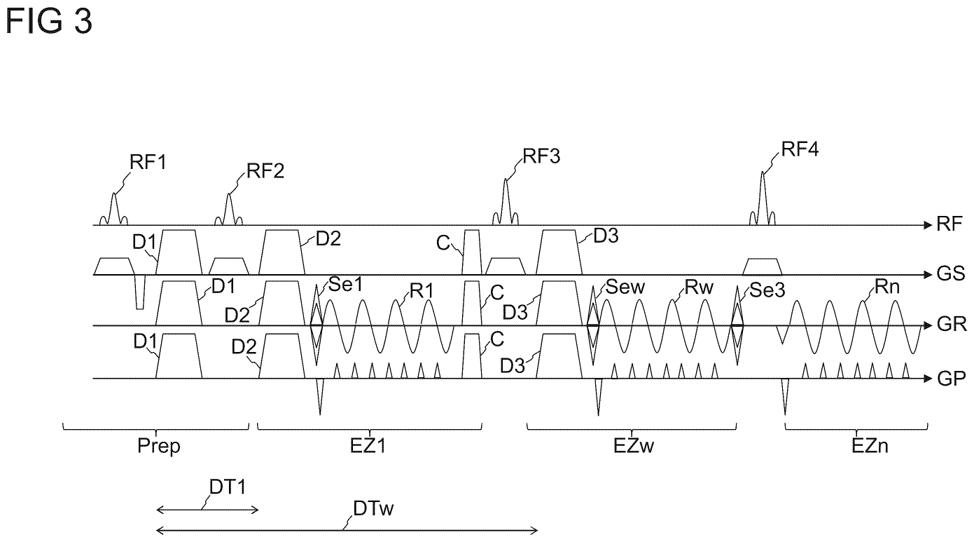

For illustration, a schematically represented portion of a pulse sequence scheme for the acquisition according to the disclosure of measurement data is shown in FIG. 3.

In the top line RF, possible RF pulses that are to be switched RF1, RF2, RF3, RF4 are represented in their temporal sequence and in relation to gradients switched in the slice selection direction GS, in the readout direction GR and in the phase encoding direction GP. For example, in a known manner, gradients can be switched in the slice-selection direction GS for a slice selection simultaneously with radiated-in RF pulses RF1, RF2, RF3, and/or RF4, which can restrict the effect of the respective RF pulse to a desired slice.

For a recording of a first formed echo signal according to an echo-planar recording technique, for example, a bipolar readout gradient R1 can be used in the readout direction GR with associated gradient blips that are to be switched in the course thereof in the phase encoding direction GP, in order to acquire measurement data of a first measurement dataset MDS1.

Similarly, for a recording of a further formed echo signal according to an echo-planar recording technique, for example, a bipolar readout gradient Rw can be used in the readout direction GR with associated gradient blips that are to be switched in the course thereof in the phase encoding direction GP, in order to acquire measurement data of a first measurement dataset MDSw.

The first and second RF pulses RF1 and RF2 described above that are to be radiated can be designated, together with the diffusion-dephasing gradient D1, which is shown here as switched in all the encoding directions GS, GR, GD, but can be switched in any desired diffusion direction, a preparation module Prep.

A switching of a first diffusion-rephasing gradient D2 and the subsequent recording according to the blocks 105 and 106 can be designated a module EZ1 of a first echo signal, and a switching of a further diffusion-rephasing gradient D3 and the subsequent recording according to the blocks 109 and 110 can be designated a module EZw of a further echo signal.

The blocks 107 to 110 can be repeated at least once directly, one after the other, wherein a third RF pulse RF3, at least on a first execution of block 107, leave a portion of the magnetization displaced by the second RF pulse RF2 into the longitudinal axis therein, so that a third RF pulse RF3, on a subsequent execution of block 107, tilts at least a portion of the magnetization displaced by the second RF pulse RF2 into the longitudinal axis, and left therein by a preceding third RF pulse RF3, into the transverse plane. For this purpose, a request 121 can be provided, which checks, for example, whether the blocks 107 to 110 are to be performed multiple times after a first RF excitation pulse RF1 and, if so, provides that all the desired repetitions are carried out.

In the example of FIG. 3, for this purpose, a further, for example, third RF pulse RF3 following the recording of the measurement data of a further measurement dataset MDSw, would be radiated in and a further module of a further echo signal EZw carried out. According to the highest diffusion value (b-value) aimed at and/or according to the highest repetition time TR aimed at, the number of support sites, for example, for a calculation of ADC maps and/or an SNR efficiency, can be increased.

For further echo signals and corresponding further measurement datasets MDSw, the above-described relationship to a further diffusion value (b-value) applies, wherein the duration DTw between the pair of diffusion-dephasing gradient D1 and (repeated) further diffusion-rephasing gradient becomes prolonged accordingly, so that by way of later repetitions of the blocks 107 to 110, larger b-values are achieved after a common first RF pulse RF1.

After the recording of at least one further echo signal as measurement data of a further measurement dataset MDSw, a center selection gradient Se3 can be switched in the readout direction (block 111), which selects a central segment, in the readout direction of the at least three segments and subsequently a fourth RF pulse RF4 can be radiated in (block 112), which refocuses the further echo signal to a third echo signal, which can be recorded in the central segment selected by the center selection gradient according to an echo-planar recording technique and can be acquired as navigator data NAVw to the measurement data recorded immediately previously of the further measurement dataset MDSw of the further echo signal (block 113). As a fourth RF pulse RF4 of this type, for example, an R-refocusing pulse can be used.

For this purpose, a request 121 can additionally or alternatively check whether the blocks 111 to 113 should be carried out and navigator data NAVw acquired to a further measurement dataset MDSw and, if so, initiate an execution of the blocks 111 to 113. Whether navigator data is to be acquired and, if so, for which further measurement datasets MDSw can be stipulated, for example, in advance by a user input or can take place automatically for all repetitions or every Nth (N being a natural number) or every Kth (K being a natural number) repetition, performed directly one after the other, of the blocks 107 to 110.

A recording of navigator data NAVw according to a block 113 can be designated a module EZn of a third echo signal to be acquired as navigator data.

Thus, following each or selected recordings of further echo signals as measurement data of a further measurement dataset MDSw, that is, after each or selected executions of the blocks 107 to 110, a center selection gradient Se3 is switched, and a fourth RF pulse RF4 is radiated in and a module EZn of a third echo signal to be acquired as navigator data is carried out. This can be advantageous, in particular, if a plurality of further measurement datasets MDSw with different diffusion values and/or if a lowest desired diffusion value lies significantly above 50, since by way of navigator data NAVw acquired in this way directly following measurement data of further measurement datasets MDSw that is to be corrected, a phase correction between segments of the respective further measurement datasets MDSw can be further improved.

On the basis of acquired navigator data NAVw, a phase correction of acquired measurement data MDSw can be carried out (block 114) in a manner that is per se known (see, e.g., the introduction of the description) in order to obtain phase-corrected further measurement datasets MDSw′.

Alternatively, or additionally, navigator data NAVw can be used in order to identify compressed measurement data of further measurement datasets MDSw (block 114). By way of the recording of third echo signals and acquisition as navigator data NAVw directly following measurement data to be tested with regard to corruption from further measurement datasets MDSw, a recognition of corrupted measurement data and thus of segments to be re-recorded can be improved. If, for example, only one set of navigator data NAVw belonging to a particular segment of a further measurement dataset MDSw of a plurality of sets of navigator data NAVw acquired following a common first RF pulse RF1 identifies the measurement data of this particular segment of the further measurement dataset MDSw as corrupted (but not all the further measurement data acquired, for example, in other segments), only for this particular segment of the further measurement dataset MDSw does measurement data need to be re-recorded. If, however, in another case, all the navigator data NAVw of the further measurement datasets MDSw acquired following a common first RF pulse RF1 identify all the measurement data of the further measurement datasets MDSw as corrupted, it can be assumed that an echo signal responsible for the corruption already recorded during the first diffusion encoding by the diffusion-dephasing gradient D1 and all the echo signals recorded after this diffusion-dephasing gradient D1 should be re-recorded.

With a request 122, it can be checked whether corrupted measurement data has been identified in further measurement datasets MDSw, and if so, a renewed recording of corresponding further echo signals can be initiated.

Therein, further echo signals from measurement data of further measurement datasets that are identified as corrupt can be re-recorded, in particular, in a renewed execution of the blocks 101 to 110 and stored again and/or further processed.

The blocks 101 to 110 (and a storage and/or further processing of the first and second measurement datasets MDS2 and MDSw thereby acquired) can be repeated with different segment selection gradients Se1, Sew so often until measurement data of the first measurement dataset MDS1 has been acquired in all the desired segments and all the further measurement datasets MDSw have been acquired. For this, a request 123 can be provided, which checks, for example, whether measurement data of the first dataset MDS1 and of all the further measurement datasets MDSw has been acquired in all the desired segments and, if not, ensures that repetitions of the blocks 101 to 110 are carried out until measurement data of the first and further measurement datasets MDS1, MDSw has been acquired in all the segments.

Therein, the first and the further segment selection gradient Se1 and Sew can select different segments of the at least three segments in at least one execution of blocks 101 to 110. This is useful, above all, if, for example, corrupted measurement data of particular segments has been identified in further measurement datasets and measurement data is to be acquired anew only in these particular segments. By way of such a variation of the recorded segments between the respective modules of first or further echo signals EZ1, EZw, for example, in a re-acquisition portion at the end of the imaging sequence in which measurement data identified as corrupted is to be acquired anew, the new recording of the segments identified as damaged can be configured particularly efficiently with previously only corrupted existing measurement data.

The blocks 101 to 110 (and a storage and/or further processing of the first and further measurement datasets MDS2 and MDSw thereby acquired) can be repeated with different diffusion-dephasing gradients D1 and first and further diffusion-rephasing gradients D2, D3 of different diffusion strengths and different desired diffusion directions so often until for all the desired diffusion strengths and diffusion directions, measurement data has been acquired in a first or further measurement dataset MDS1, MDSw. For this, a request 123 can additionally or alternatively check whether for all the desired diffusion strengths and diffusion directions, measurement data has been acquired in a first or further measurement dataset MDS1, MDSw and, if not, an initiation takes place of repeated execution of the blocks 101-110 until measurement data has been acquired in a first or further measurement dataset MDS1, MDSw for all the desired diffusion strengths and diffusion directions.

In this way, for example, further diffusion values can be generated, according to which first and further measurement datasets MDS1, MDSw are diffusion weighted. Further diffusion values can herein be determined again, for example, according to b=γ² G² δ² (DT1−δ/3) and/or according to b=γ² G² δ² (DTw−δ/3), where γ is the gyromagnetic ratio, G is the gradient amplitude of the respective gradients (see above), δ is the gradient duration of the respective gradients (see above), and DT1 and/or DTw is the duration between the pair of diffusion-dephasing gradient D1 and first diffusion-rephasing gradient D1 and/or between the pair of diffusion-dephasing gradient D1 and further diffusion-rephasing gradient D3.

Following the recording of a first echo signal, at least one crusher gradient C can be switched in at least one encoding direction and dephases a still-existing signal of the first echo signal before a third RF pulse RF3 is radiated in.

An increase of modules of further echo signals carried out after a first RF pulse RF1 and/or third echo signals and thus a prolongation of the repetition time TR can be useful, in particular, in combination with SMS recording techniques, since there an achievable reduction in the repetition time TR is often already limited by the necessary image contrast and thus this can possibly be optimally exploited first through an increase of modules of further echo signals and/or of third echo signals carried out after a first RF pulse RF1.

The proposed method offers the advantage of measuring time shortening with echo-planar recording techniques that are segmented in the readout direction, such as for example, RESOLVE sequences. By way of the temporally closely associated recording of measurement datasets MDS1, MDSw with different diffusion weighting, otherwise detrimental influences by way of a movement, including a breathing movement, can be reduced.

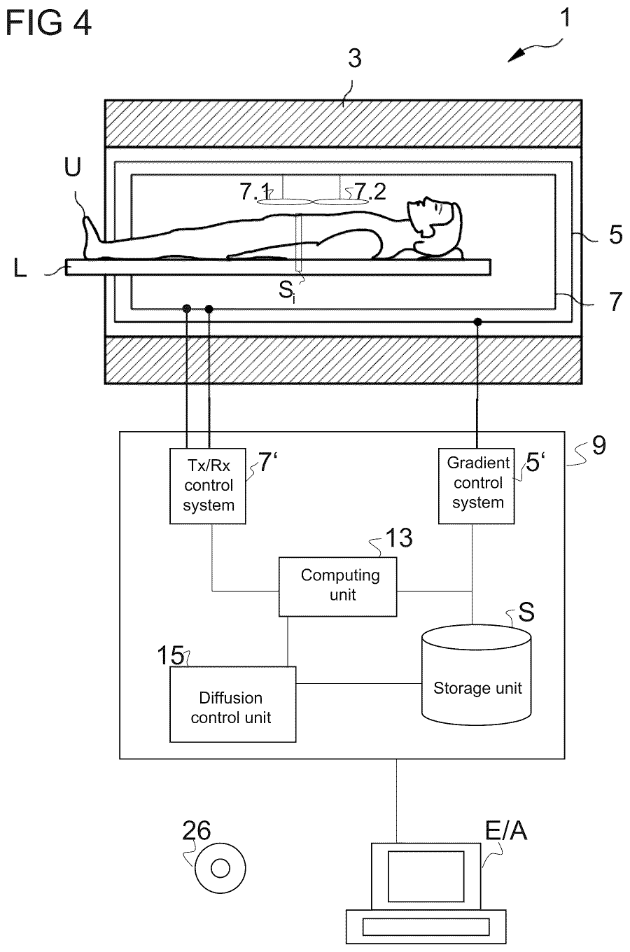

FIG. 4 schematically represents a magnetic resonance system 1 according to the disclosure. It comprises a magnet unit 3 for generating the main magnetic field, a gradient unit 5 for generating the gradient fields, a high frequency unit 7 for radiating in and receiving high frequency signals, and a control apparatus 9 configured for carrying out a method according to the disclosure.

In FIG. 4, these subunits of the magnetic resonance system 1 are shown only roughly schematically. In particular, the high frequency unit 7 can consist of a plurality of subunits, for example, a plurality of coils such as the schematically shown coils 7.1 and 7.2 or more coils, which can be configured either only to transmit high frequency signals or only to receive the triggered high frequency signals or for both.

For investigation of an examination object U, for example, a patient or a phantom, it can be introduced on a support L into the magnetic resonance system 1, in the measuring volume thereof. The slice or the slab Sᵢ represents an exemplary target volume of the examination object from which echo signals are to be recorded and captured as measurement data.

The control apparatus 9 serves to control the magnetic resonance system 1 and can, in particular, control the gradient unit 5 by means of a gradient control system 5′ and the high frequency unit 7 by means of a high frequency transmitting/receiving control system 7′. The high frequency unit 7 can herein comprise a plurality of channels on which signals can be transmitted or received.

The high frequency unit 7 is responsible, together with its high frequency transmitting/receiving control system 7′ for the generation and radiating-in (transmission) of a high frequency alternating field for manipulation of the spins in a region to be manipulated (for example, in slices S to be scanned) of the examination object U. Herein, the center frequency of the high frequency alternating field, also designated the B1 field, is typically adjusted so that, as far as possible, it lies close to the resonance frequency of the spin to be manipulated. Deviations of the center frequency from the resonance frequency are referred to as off-resonance. In order to generate the B1 field, in the high frequency unit 7, currents controlled by means of the high frequency transmitting/receiving control system 7′ are applied to the HF coils.

Furthermore, the control apparatus 9 comprises a diffusion control unit 15 with which RF pulses and gradients according to the disclosure for diffusion preparation of measurement data can be determined, which pulses and gradients can be implemented by the gradient control system 5′ and the high frequency transmitting/receiving control system 7′. The control apparatus 9 is configured overall to carry out a method according to the disclosure.

A computing unit 13 included by the control apparatus 9 is configured to carry out all the computation operations necessary for the required measurements and determinations. Intermediate results and results needed for this or established herein can be stored in a storage unit S of the control apparatus 9. The units mentioned are herein not necessarily to be understood as physically separate units but merely represent a subdivision into units of purpose which, however, can also be realized, for example, in fewer or even only in one single physical unit.

Via an input/output apparatus E/A of the magnetic resonance system 1, for example, control commands can be passed, for example, by a user to the magnetic resonance system, and/or results from the control apparatus 9 such as, for example, image data can be displayed.

A method described herein can also exist in the form of a computer program product, which comprises commands that carry out the described method on a control apparatus 9. Similarly, a computer-readable storage medium can be provided, which comprises commands that, when executed by a control apparatus 9 of a magnetic resonance system 1, cause it to carry out the method described.

Independent of the grammatical term usage, individuals with male, female, or other gender identities are included within the term.

1. A method for optimized recording of diffusion-weighted measurement data of an examination object using a magnetic resonance system, comprising:
a) radiating in a first RF pulse, which tilts a longitudinal magnetization into a transverse plane;
b) switching a diffusion-dephasing gradient of a desired diffusion strength in a desired diffusion direction following the first RF pulse;
c) radiating in a second RF pulse, which displaces a portion of the magnetization situated in the transverse plane back again into a longitudinal axis and leaves another portion of the magnetization situated in the transverse plane therein;
d) switching a first diffusion-rephasing gradient in the desired diffusion direction, which rephases the magnetization remaining in the transverse plane so that after the first diffusion-rephasing gradient, a first echo signal is formed;
e) switching a segment-selection gradient in a readout direction for selection of one of at least three segments;
f) recording the first echo signal in the segment selected by way of the segment selection gradient as measurement data according to an echo-planar recording technique and acquiring it as measurement data of a first measurement dataset;
g) following the recording of the first echo signal, radiating in a third RF pulse, which tilts at least a portion of the magnetization displaced back into the longitudinal axis by way of the second RF pulse into the transverse plane;
h) switching a further diffusion-rephasing gradient in the desired diffusion direction, which rephases the magnetization tilted by the third RF pulse into the transverse plane so that after the further diffusion-rephasing gradient, a further echo signal is formed;
i) switching a further segment-selection gradient in the readout direction for selection of one of the at least three segments;
j) recording the further echo signal in the segment selected by way of the further segment selection gradient as measurement data according to an echo-planar recording technique and acquiring it as measurement data of a further measurement dataset; and
k) storing and/or further processing the acquired first measurement dataset and acquired further measurement datasets.

2. The method as claimed in claim 1, wherein a first echo signal is diffusion-prepared in the desired diffusion direction with a first diffusion value according to the previously switched diffusion-dephasing gradient and the first diffusion-rephasing gradient and a first timespan elapsed between the switched diffusion-dephasing gradient and the first diffusion-rephasing gradient, and the measurement data of the first measurement dataset is diffusion-weighted according to the first diffusion value, and wherein a further echo signal is diffusion-prepared in the desired diffusion direction with a further diffusion value according to the previously switched diffusion-dephasing gradient and the further diffusion-rephasing gradient and the further timespan elapsed between the switched diffusion-dephasing gradient and the further diffusion-rephasing gradient, and the measurement data of further measurement datasets is diffusion-weighted according to the respective further diffusion value.

3. The method as claimed in claim 1, wherein the steps g) to j) are repeated at least once directly one after the other, wherein a third RF pulse, at least on a first execution of step g), leaves a portion of the magnetization displaced by the second RF pulse into the longitudinal axis therein, so that a third RF pulse, on a subsequent execution of step g), tilts at least a portion of the magnetization displaced by the second RF pulse into the longitudinal axis and left therein by a preceding third RF pulse, into the transverse plane.

4. The method as claimed in claim 1, wherein after the recording of at least one further echo signal, a center selection gradient is switched in the readout direction, which selects a central segment, in the readout direction, of the at least three segments, and subsequently a fourth RF pulse is radiated in which refocuses the further echo signal to a third echo signal, which is recorded in the central segment selected by the center selection gradient according to an echo-planar recording technique and is acquired as navigator data to the further measurement dataset of the further echo signal.

5. The method as claimed in claim 4, wherein based on acquired navigator data, a phase correction of acquired measurement data is carried out.

6. The method as claimed in claim 4, wherein navigator data is used to identify corrupted measurement data of further measurement datasets.

7. The method as claimed in claim 6, wherein further echo signals from measurement data of further measurement datasets identified as corrupt are re-recorded in a renewed execution of steps a) to k).

8. The method as claimed in claim 1, wherein steps a) to k) are repeated with different segment selection gradients so often until, in all desired segments, measurement data of the first measurement dataset and of all the desired further measurement datasets has been recorded.

9. The method as claimed in claim 1, wherein a first and the further segment selection gradient select different segments of the at least three segments in at least one execution of steps a) to k).

10. The method as claimed in claim 1, wherein steps a) to k) are repeated with different diffusion-dephasing gradients and first and further diffusion-rephasing gradients of different diffusion strengths and different desired diffusion directions so often until, for all the desired diffusion strengths and diffusion directions, measurement data has been recorded in a first or further measurement dataset.

11. The method as claimed in claim 1, wherein following the recording of a first echo signal, at least one crusher gradient is switched in at least one encoding direction, which dephases a still existing signal of the first echo signal.

12. A magnetic resonance system, comprising:
a magnet unit;
a gradient unit;
a high frequency unit; and
a control apparatus with a high frequency transmitting/receiving control system and a diffusion control unit, wherein the control apparatus is configured to carry out the method as claimed in claim 1 on the magnetic resonance system.

13. A non-transitory computer-readable storage medium comprising commands which, on execution by a control apparatus of a magnetic resonance system, cause the magnetic resonance system to carry out the method as claimed in claim 1.
